(19) United States
(12) Patent Application Publication
(10) Pub. No.: US 2020/0357491 A1
(43) Pub. Date: Nov. 12, 2020

(54) METHODOLOGY FOR MEASURING THE QUALITY OF PHYLOGENETIC AND TRANSMISSION TREES AND FOR MERGING TREES

(71) Applicant: KONINKLIJKE PHILIPS N.V., EINDHOVEN (NL)

(72) Inventors: HENRY LIN, QUINCY, MA (US); BRIAN DAVID GROSS, NORTH ANDOVER, MA (US); KARTHIKEYAN MURUGESAN, CAMBRIDGE, MA (US)

(21) Appl. No.: 16/760,213

(22) PCT Filed: Dec. 10, 2018
(86) PCT No.: PCT/EP2018/084074

§ 371 Date: Apr. 29, 2020

Related U.S. Application Data
(60) Provisional application No. 62/598,587, filed on Dec. 14, 2017.

(51) Int. Cl. G16H 10/20; G16H 50/80

(52) U.S. Cl. CPC G16H 10/20; G16H 50/80

(57) ABSTRACT

In healthcare associated infection (HAI) outbreak tracking, different transmission tree inference algorithm processes (40) are performed on genetic variants data (26) for a set of HAI infected persons to generate a plurality of transmission trees (42) representing parent-child infectious transmission links. For each transmission tree, the value (44) of a correlation metric is computed, which measures correlation of the transmission tree with a clinical correlate (46). For each random trial of a plurality of random trials (52), the value (54) of the correlation metric is also computed. A statistical likelihood (60) of each transmission tree given the clinical correlate is estimated from the computed values of the correlation metric for the random trials and for the transmission tree. This may, for example, be a p-value. An optimal transmission tree is selected from amongst the plurality of transmission trees based on the estimated statistical likelihoods.

### FIELD

The following relates generally to the healthcare associated infection (HAI) outbreak tracking arts, HAI transmission tree inference arts, genetic sequencing arts, and related arts.

### BACKGROUND

Healthcare-associated infections (HAIs) are patient acquired infections received during healthcare treatments for different conditions. HAIs in the medical literature are referred to as nosocomial infections. HAIs can be deadly and are a frequent occurrence in hospitals. They include bacterial or fungal causes. In some estimates, approximately one out of every twenty hospitalized patients will contract an HAI, and this is an issue in both Europe and the United States, as well as other geographical regions.

Prevention of the spread of HAIs is the first line of defense, with techniques such as sanitation/sterilization, handwashing, use of gloves or other barrier mechanisms, and so forth being effective tools for reducing HAI transmission.

When an HAI outbreak is detected, the task turns to tracing the transmission path so as to identify and treat all persons exposed to the contagion. Measures such as quarantine of both symptomatic and asymptomatic persons exposed to the contagion are taken to prevent further spread. The traditional approach for tracing the transmission path is the labor-intensive process of identifying infected persons and identifying the transmission pathways. Depending on the type of infectious agent, transmission pathways may include contact transmission, droplet transmission (i.e. transmission via droplets expelled during sneezing or coughing), airborne transmission, surface-mediated transmission, transmission via contaminated food or water, or so forth. By interviewing infected persons or other investigative means, clinical correlates are identified which are potential transmission pathways linking infected persons. These clinical correlates are leveraged to identify parent-child relationships in which the “parent” infected person transmits the infection to the “child” infected person. These form a transmission tree, and the goal is to trace the infection pathways backward to the original source (e.g. a contaminated food source, or a “patient zero”, or so forth). This traditional approach is time consuming and prone to error due to inaccurate recollections of interviewed infected persons or the like, failure to identify some infected persons (especially in the case of asymptomatic infected persons who may not seek medical attention yet can act as undetected transmission vectors), or uncooperative infected persons.

More recently, genomic sequencing has been leveraged to perform tracking of transmission pathways in HAI outbreaks. This approach employs genomic sequencing of bacterial, fungal, or other HAI contagion isolates drawn from infected persons. The approach leverages the rise of next generation sequencing (NGS) which is capable of rapidly producing a whole genome sequence (WGS), whole exome sequence (WES), or other genetic sequence for the isolate in a time frame on the order of hours or shorter. The approach further leverages the rapid phylogenetic diversification of typical HAI contagions which leads to introduction of genetic variants on the scale of single transmission events. Hence, the introduced genetic variants are traceable from one infected person to the next, enabling a transmission tree to be generated by comparing the population of genetic variants in isolates drawn from different HAI-infected persons. Advantageously, the genomic sequencing approach for generating the transmission tree is not dependent upon subjective and error-prone personal recollections of recent activities, and can detect transmission pathways even when an intervening vector remains undetected. As an example of the latter benefit, consider the illustrative case of transmission from person A to person B to person C, where person B is an undetected asymptomatic person who unwittingly served as the vector for transmission from person A to person C. Even without detecting person B, comparison of the variants of the isolates drawn from persons A and C may establish that person C was infected from person A.

One difficulty with using genomic sequencing for tracing HAI transmission pathways is the large computational complexity entailed in processing the variants of the different isolates to detect parent/child transmission relationships. In general, a phylogenetic tree is reconstructed from variants data of the isolates. The phylogenetic tree captures the evolutionary relationships of the isolates. It is generally straightforward to transform the phylogenetic tree into a transmission tree, although some ambiguities can arise during this transformation, e.g. the isolates drawn from two or more persons may be so genetically similar that it may not be possible to unambiguously assign parent/child transmission relationships between these persons on the basis of the genetic sequencing. Some known phylogenetic inference tools for reconstructing a phylogenetic or transmission tree from variants data of the isolates include, by way of non-limiting illustration, distance matrix-based methods, RAxML and variants thereof available from The Exelixis Lab, Heidelberg, Germany which employ maximum likelihood inference methods; minimum spanning tree (MST) based inference methods, or so forth.

The following discloses a new and improved systems and methods.

### SUMMARY

In one disclosed aspect, a non-transitory storage medium stores instructions readable and executable by an electronic processor to perform a healthcare associated infection (HAI) outbreak tracking method. In the method, a plurality of transmission tree inference algorithm processes are performed, operating on genetic variants data for a set of HAI infected persons, to generate a plurality of transmission trees representing parent-child infectious transmission links between pairs of HAI infected persons. For each transmission tree, the value of a correlation metric is computed which measures correlation of the transmission tree with a clinical correlate. For each random trial of a plurality of random trials each comprising parent-child links randomly generated between pairs of HAI infected persons of the set of HAI infected persons, the value of the correlation metric is similarly computed. A statistical likelihood of each transmission tree is estimated given the clinical correlate from the computed values of the correlation metric for the random trials and for the transmission tree. The statistical likelihood may be an estimated p-value, for example. At least one transmission tree of the plurality of transmission trees is displayed. The displayed at least one transmission tree is at least one of (i) selected for display based on the estimated statistical likelihoods or (ii) labeled with the estimated statistical likelihoods.

In another disclosed aspect, a device is disclosed for performing HAI outbreak tracking. The device comprises a computer, a display operatively connected with the computer, and a non-transitory storage medium as set forth in the immediately preceding paragraph. The computer is operatively connected to read and execute the instructions stored on the non-transitory storage medium to perform the HAI outbreak tracking method.

In another disclosed aspect, a device is disclosed for performing HAI outbreak tracking. The device comprises a computer, a display operatively connected with the computer, and a non-transitory storage medium storing instructions readable and executable by the computer to perform an HAI outbreak tracking method. This method includes: performing a plurality of transmission tree inference algorithm processes operating on genetic variants data for a set of HAI infected persons to generate a plurality of transmission trees representing parent child infectious transmission links between pairs of HAI infected persons; computing statistical likelihoods of parent child infectious transmission links in the transmission trees based on at least one of correlation with one or more clinical correlates and frequency of occurrence of the links in the plurality of transmission trees; identifying one or more low confidence parent child infectious transmission links based on the computed statistical likelihoods; and displaying, on the display, at least one transmission tree selected from or derived from the plurality of transmission trees wherein the displaying includes graphically indicating the one or more low confidence parent child infectious transmission links in the display of the at least one transmission tree.

In another disclosed aspect, a method of HAI outbreak tracking comprises the operations (i), (ii), (iii), (iv), (v), and (vi). Operation (i) performs a plurality of transmission tree inference algorithm processes operating on genetic variants data for a set of HAI infected persons to generate a plurality of transmission trees representing parent child infectious transmission links between pairs of HAI infected persons. In operation (ii), for each transmission tree, the value is computed of a correlation metric measuring correlation of the transmission tree with a clinical correlate. In operation (iii), for each random trial of a plurality of random trials each comprising parent-child links randomly generated between pairs of HAI infected persons of the set of HAI infected persons, the value is also computed of the correlation metric. Operation (iv) estimates a statistical likelihood of each transmission tree given the clinical correlate from the computed values of the correlation metric for the random trials and for the transmission tree. Operation (v) selects an optimal transmission tree from amongst the plurality of transmission trees based on the estimated statistical likelihoods of the trees given the clinical correlate. Operation (vi) displays the optimal transmission tree on a display. Operations (i), (ii), (iii), (iv), and (v) are suitably performed by a computer executing instructions stored on a non-transitory storage medium.

One advantage resides in providing healthcare associated infection (HAI) outbreak tracking using transmission trees inferred from genomic data of HAI infected persons, which leverages transmission trees inferred using different transmission tree inference processes to display a transmission tree having a higher statistical likelihood of correlating with actual transmission pathways of the HAI outbreak.

Another advantage resides in providing HAI outbreak tracking using one or more transmission trees inferred from genomic data of HAI infected persons, which provides graphical indication of low confidence parent-child infection transmission links.

Another advantage resides in providing either one or both of the foregoing benefits with synergistic leveraging a plurality of different clinical correlates.

Another advantage resides in providing one or more of the foregoing benefits tuned to specific characteristics of the known or suspected pathogen causing the HAI.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a device for performing healthcare associated infection (HAI) outbreak tracking using genomic sequencing data collected from HAI infected persons.

FIG. 2 diagrammatically illustrates three transmission trees inferred by different transmission tree inference algorithm processes, in which parent-child infectious transmission links to a node P3 have low confidence.

FIGS. 3 and 4 illustrate two possible approaches for displaying a transmission tree for the nodes of FIG. 2 with graphical indication of the low confidence parent-child infectious transmission links to the node P3.

### DETAILED DESCRIPTION

As previously mentioned, various algorithms are available for reconstructing a phylogenetic or transmission tree from variants data of HAI contagion isolates drawn from infected persons. However, these algorithms sometimes produce different and inconsistent results. Even using different tuning parameters for the same algorithm can produce different and inconsistent transmission trees. In general, isolates with low single nucleotide polymorphism (SNP) variant scores can lead to errors in the reconstructed tree as the parent-child relationships may flip randomly and generate erroneous apparent lineage relationships based on random noise and other non-deterministic causes.

Furthermore, reconstruction of transmission tree from genomic variants data fails to leverage clinical correlates, such as location history, caretaker information, equipment or procedure usage, or so forth, which may provide a rational basis for deducing transmission pathways from one infected person to another. For example, if the pathogen is transmittable via contaminated surfaces and a medical device was used for infected patient A and then later was used for infected patient B (within the surface residency time of the pathogen) then it may be rationally suspected that patient B was infected from patient A via the transmission vector of contaminated surfaces of the medical instrument. As another example, if nurse X treated patient A and then treated patient B a similar rational suspicion may arise under the hypothesis that nurse X was a transmission vector, especially if nurse X is also determined to have been infected and contagious. Clinical correlates may be leveraged on an ad hoc basis, e.g. if an emergency management specialist is suspicious that a parent-child link in a transmission tree generated from genomic data may be in error, then the specialist might elect to replace the suspicious link with an alternative transmission pathway deduced from a clinical correlate. However, this ad hoc approach does not provide a principled or systematic way for integrating clinical correlate data to improve the transmission tree.

In another approach, the “quality” of the transmission tree can be assessed by quantifying how well the transmission tree agrees with transmission predicted by a clinical correlate. For example, the number of edges of a transmission tree produced by genomic analyses that match with transmissions deduced from the clinical correlate may be counted to provide a quantitative measure of agreement. A high count may provide more confidence in the validity of the transmission tree. However, the count of matches is a rough estimate that may be insufficient to choose between two or more inconsistent transmission trees generated by different genomic analysis algorithms (or by the same algorithm with different tuning) For example, the clinical correlate is usually insufficient to reconstruct a full transmission tree, so the clinical correlate may provide no information as to accuracy of many edges of the phylogenetically produced transmission tree may. More generally, the count of matches does not provide a strong basis for improving upon the transmission tree or trees provided by the one or more genomic analysis algorithms.

In embodiments disclosed herein, selection of a transmission tree from amongst a plurality of generated trees is performed by comparing correlation of the transmission tree with a clinical correlate against the null hypothesis. In an illustrative approach, this is done by computing a correlation metric measuring how well a transmission tree correlates with the clinical correlate; the same correlation metric is computed for a set of random trials, and a p-value is estimated as the fraction of random trials that correlate with the clinical correlate better than the transmission tree. The transmission tree having the lowest p-value is then selected. In a variant embodiment, similar comparison against the null hypothesis is performed on a per-parent-child link basis, and these statistics are used to select the best links from amongst several transmission trees to generate a merged transmission tree. Additionally or alternatively, these statistics may be used to display the transmission tree using link representations indicative of their statistical confidence.

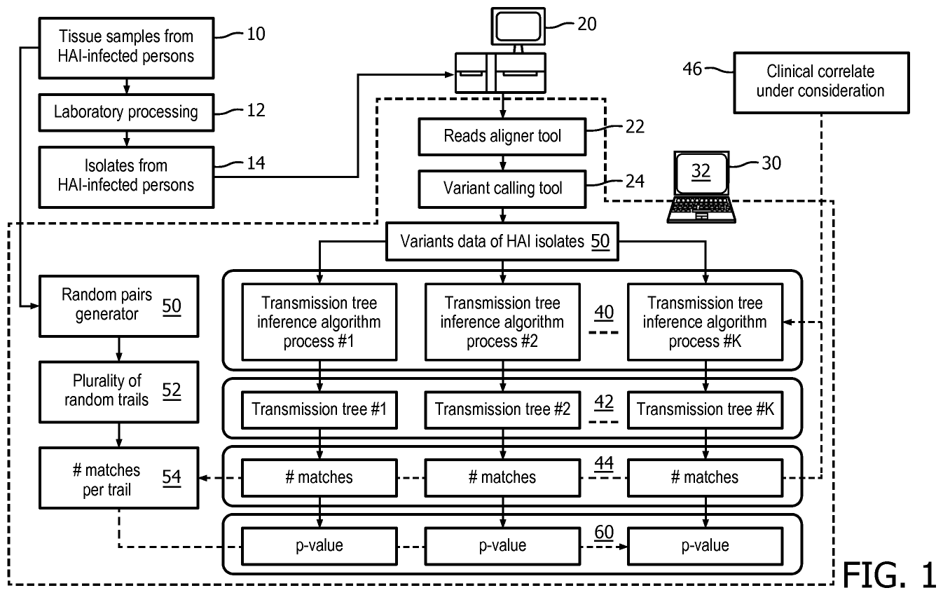

With reference to FIG. 1, an illustrative system employing genomic sequencing for tracing HAI transmission pathways is shown. A clinician draws tissue samples 10 from HAI-infected persons, e.g. as a drawn blood sample, buccal smear, or so forth. Laboratory processing 12 is performed on the tissue samples to isolate the infectious (or suspected infectious) pathogen, thereby generating isolates 14 drawn from the infected persons. The choice of type of tissue sample 10 and the type(s) of the laboratory processing 12 depend upon the type of infectious agent known or suspected to be responsible for the HAI outbreak. For example, in some known approaches the tissue samples 10 are cultured using nutrient substrates or media reasonably expected to promote growth of the known or suspected pathogen. Where the pathogen is unknown, multiple types of tissue samples may be initially drawn and variously cultured in an effort to isolate and identify the responsible pathogen. In addition to pathogen isolation, the laboratory processing 12 also prepares the sample for genetic sequencing. For example, the laboratory processing 12 may include various sample preparation known in the art, e.g. wet lab procedures to extract purified deoxyribonucleic acid (DNA) from the sample, perform end repair/modification, polymerase chain reaction (PCR) amplification, and so forth.

The resulting isolate samples 14 are loaded into a genetic sequencer 20, typically using sample cartridges designed for this purpose. The genetic sequencer 20 operates to generate unaligned DNA sequence fragment reads, that is, data representations of base sequences of DNA fragments, preferably with read confidence (i.e. “quality”) scores for the bases of the sequence. The DNA fragment reads may, for example, be stored in the commercially common FASTQ format. By way of non-limiting illustrative example, the genetic sequencer 20 may, for example, comprise an Illumina™, PacBio™, Ion Torrent™, Nanopores™, ABI-SOLiD™, or other commercially available genetic sequencer. The DNA preparatory component of the laboratory processing 12 is typically tailored to the chosen genetic sequencer 20 and is performed in accordance with procedures promulgated by the sequencer manufacturer and, in some instances, using proprietary chemicals provided by the sequencer manufacturer. Depending upon the choice of processing, the DNA sample and consequently the reads may be limited to a particular type or selection of DNA, e.g. selective PCR may be used to selectively amplify only certain DNA portions. For example, only certain genes (i.e., protein-encoding exons) may be sequenced, by using known target enrichment processing to isolate the selected exons. If the DNA isolation/amplification processing is not selective, then all DNA material of the isolate is amplified, thus providing for whole genome sequencing (WGS).

The unaligned reads are aligned or mapped by a reads aligner/mapper tool 22 to a reference sequence for the known or suspected pathogen (or the amplified portions thereof) to generate an aligned DNA sequence. By way of non-limiting illustrative example, the reads aligner tool 22 may for example comprise a Burrows-Wheeler Alignment (BWA) tool for performing short read alignment followed by a processing by the SAMtools suite to align longer sequences. The resulting aligned sequence may, for example, be stored in a commercially standard Sequence Alignment/Map (SAM) or Binary Alignment Map (BAM) format. A variant calling tool 24 employs suitable approaches for identifying genetic variants in the aligned DNA sequence. The genetic variants may be single nucleotide substitution variants, sometimes referred to as single nucleotide polymorphism (SNP) or single nucleotide variant (SNV) variants; base modification variants (e.g. methylation), an “extra” inserted base or a missing, i.e. “deleted” base, commonly referred to collectively as indels, copy number variations (CNVs), or so forth. In a suitable approach, the variant caller 24 calls genetic variants contained in the DNA sequence as compared with the reference DNA sequence. To account for low read coverage and other complications, the variant caller 24 may employ probabilistic or statistical methods for identifying genetic variants. It will be appreciated that the sequencing, reads alignment, and variant calling are performed for each HAI isolate 14 (that is, for the pathogen isolate extracted from each HAI-infected person undergoing testing) to produce variants data 26 for the HAI isolates. The resulting variants data 26 may comprise a list of genetic variants for each isolate which is stored in a standard variant calls file (VCF) format.

With continuing reference to FIG. 1, the various processing components, e.g. the reads aligner 22, variant caller 24, and various transmission tree inference and scoring components to be described in the following, are suitably implemented on a computer or other electronic processor 30 which reads and executes instructions stored on a non-transitory storage medium, which instructions when executed by the electronic processor 30 implement the various computational components, e.g. the reads aligner 22, variant caller 24, and various transmission tree inference and scoring components to be described. While the illustrative electronic processor 30 is a desktop computer, it may alternatively or additionally comprise a server computer, a cluster of server computers, a distributed computing resource in which electronic processors are operatively combined on an ad hoc basis (e.g. a cloud computing resource), an electronic processor of the genetic sequencer 10, and/or so forth. The non-transitory storage medium storing the instructions which are read and executed by the electronic processor 30 may, for example, comprise one or more of: a hard disk drive or other magnetic storage medium; a flash memory, solid state drive (SSD), or other electronic storage medium; an optical disk or other optical storage medium; and/or so forth. Furthermore, the electronic processor 30 includes or is operatively connected with a display 32 on which the transmission tree(s) and/or other isolate and/or transmission pathway data may be displayed.

With continuing reference to FIG. 1, the variants data 26 of the HAI isolates serve as input data to a plurality of transmission tree inference algorithm processes 40, which operate to generate a corresponding plurality of transmission trees 42. Each transmission tree 42 represents parent-child infectious transmission links between pairs of HAI infected persons drawn from the set of HAI infected persons represented by the variants data 26. Without loss of generality, in FIG. 1 the number of transmission tree inference algorithm processes 40 is enumerated as K, where K is an integer greater than or equal to two, and the corresponding transmission trees 42 are likewise enumerated 1, . . . , K. With the variants data 26 for the set of sequencing samples for the HAI infected persons, pairwise distances can be computed between each pair of samples and the resulting distance matrix used to build a phylogeny or transmission tree of samples to show how outbreaks may have spread. These are called distance matrix based transmission tree inference algorithms. Some other transmission tree inference algorithms have also been developed that do not need to create a distance matrix on all samples. Besides utilizing many of the methods that have been developed like neighbor-joining, RAxML (http://sco.h-its.org/exelixis/software.html), or minimum spanning tree based methods, several methods have various model parameters that can be tuned, or SNP calling/filtering methodologies that can also create different types of phylogeny or transmission trees. In general, the transmission tree inference algorithm processes 40 may employ any phylogenetic tree inference algorithm, such as (by way of non-limiting illustration) distance matrix-based methods, RAxML and variants thereof available from The Exelixis Lab, Heidelberg, Germany which employ maximum likelihood inference methods; minimum spanning tree (MST) based inference methods, or so forth. The various transmission tree inference algorithm processes 40 may differ by employing different transmission tree inference algorithms, and/or two or more of these processes may employ the same transmission tree inference algorithm but with different tuning parameters for the transmission tree inference algorithm.

A particular transmission tree inference algorithm may operate exclusively on the variants data 26 of the HAI isolates, or may employ other information as constraints on the tree inference. For example, some transmission tree inference algorithms employ infection dates for the HAI infected persons as constraints on the transmission tree inference algorithm, e.g. if infected person A has an infection date that precedes the infection date of infected person B, then B is suitably constrained against being the parent of A in a parent-child infectious transmission link, i.e. the link B→A is prohibited. More generally, if it is known that a first person has an infection date that is later than the infection date of the second person, then a constraint may be imposed that the first person cannot be the parent of the second person (in the sense of an infectious transmission pathway). Since infection dates often have a large uncertainty, these constraints may be soft constraints—for example if A has an infection date range whose center precedes the infection date range of B but the infection date ranges overlap, then a soft constraint may be implemented to capture the reduced statistical chance of parent-child link B→A in view of these infection date ranges.

As another example, a particular transmission tree inference algorithm may employ a clinical correlate as a constraint. For example, if it is known that infected person M and infected person J both came into close proximity with a medical device whose surface is determined to have been contaminated with the HAI pathogen (or is suspected of such contamination) then this clinical correlate information may be used to enhance the likelihood of M→J or J→M in the inferred transmission tree. If the dates of contact with the medical device are also known then the clinical correlate can be thereby refined, e.g. to only support the pair M→J if person J came into proximity to the medical device after person M. In the particular phylogenetic inference algorithm, the clinical correlate may be used to increase the selection weight of those candidate parent-child infectious transmission links that are consistent with, or are made more probable in view of, the clinical correlate.

Given that many different phylogeny or transmission trees 42 can be created, it is desired to evaluate the quality of the phylogenetic or transmission trees 42 based on limited clinical data, in the absence of full information regarding true transmissions, in order to select the optimal transmission tree. Optionally, one or more low confidence parent-child infectious transmission links of a transmission tree may be identified based on statistical likelihoods computed based on at least one of correlation with one or more clinical correlates and frequency of occurrence in the plurality of transmission trees.

Clinical data that can be correlated (at least in some instances) with HAI transmission are referred to herein as clinical correlates: these can include location history, caretaker information, and equipment or procedure usage. For example, a clinical correlate may be a medical device that came into proximity with two or more HAI infected persons (in the case of an HAI that is transmittable via surface transmission), or a caregiver who came into contact with two or more HAI infected persons, or so forth.

In the illustrative approaches, matches between tree links and a clinical correlate are compared with how frequently matches would occur by random chance (e.g. taking links between two HAI infected persons randomly). By comparing the matches with the clinical correlate observed in the transmission tree and comparing with the matches observed in a set of randomly generated links, a statistic such as a p-value is associated with the transmission tree to indicate how likely the tree is to have identified transmissions over random chance. Simultaneously, this p-value can also be used as a measure of quality for the transmission tree in terms of identifying transmissions. In order to estimate the p-value, a random sampling is used to determine the number of matches expected to be seen randomly over multiple simulated trials, and it is measured how frequently this number of matches exceeds the number of matches found in the phylogenetically inferred transmission tree. The p-value is then estimated by dividing the number of times the random matches exceeds the matches seen in the transmission tree by the total number of random trials. In this analysis, the p-value is computed with the null hypothesis that the phylogenetically inferred transmission tree is random and is not informative of transmissions, while the alternative hypothesis is that the phylogenetically inferred transmission tree is informative of transmissions.

The p-value can be used to determine which transmission tree from amongst the plurality of transmission trees 42 is most likely representing the transmissions in the case of multiple phylogeny algorithms 40 being used, and can be used to indicate to the user where parent-child and lineage demarcations may be at lower confidence. An absolute confidence setting can be used to ensure consistency in what is present to the user.

With continuing reference to FIG. 1, to this end, for each transmission tree 42, the value 44 of a correlation metric is computed, which measures correlation of the transmission tree with a clinical correlate 46. In the illustrative example, the correlation metric comprises a count of parent-child infectious transmission links between pairs of HAI infected persons in the transmission tree 42 that match with the clinical correlate 46. In parallel, a random pairs generator 50 operates to generate a plurality of random trials 52. Each random trial comprises parent-child links randomly generated between pairs of HAI infected persons of the set of HAI infected persons (or analogously, of the set of tissue samples 10 from those HAI infected persons). For each random trial, the value 54 of the correlation metric is computed, which measures correlation of the random trial with the clinical correlate 46. The same correlation metric is used as in assessing the trees 42, i.e. in the illustrative example the correlation metric again comprises a count of (here randomly generated) links between pairs of HAI infected persons that match with the clinical correlate 46. A statistical likelihood 60 of each transmission tree 42 given the clinical correlate 46 is then estimated from the computed values 54 of the correlation metric for the random trials and the computed values 44 of the correlation metric for the transmission tree.

In a suitable formulation, let C_{T} represent the value 44 of the correlation metric for a transmission tree 42. For the illustrative example, C_{T} is the count of parent-child infectious transmission links between pairs of HAI infected persons in the transmission tree 42 that match with the clinical correlate 46. Further let C_{R,i} represent the value 54 of the correlation metric for the random trial indexed by i, where i=1, . . . , N and N is the total number of random trials. The estimated statistical likelihood for each transmission tree 42 comprises a p-value in the illustrative example. This p-value for the transmission tree is estimated as a fraction of the random trials 52 whose correlation with the clinical correlate 46 as measured by the correlation metric is higher than the correlation of the transmission tree 42 with the clinical correlate 46 as measured by the correlation metric. For the illustrative example using the p-value as the correlation metric and the notation given above, let a count T be the number of times the random trial yields more matches than the transmission tree can be computed, that is, the number of times where C_{R,i}>C_{T} over the random trials i=1, . . . , N. Then the p-value is given by the ratio TIN. Conceptually, it will be recognized that for a transmission tree that strongly correlates with actual transmissions (and hence should also strongly correlate with the clinical correlate 46), the number of times T that the random trial yields more matches than the transmission tree should be very low, so that the p-value TIN should be close to zero. Said another way, the p-value measures the statistical significance of the transmission tree for identifying potential transmissions (i.e. rejecting the null hypothesis that our tree is random and not informative of transmissions). Lower p-values thus indicate higher quality transmission trees which are more informative of transmissions.

The p-values 60 can be used to select an optimal transmission tree from amongst the plurality of transmission trees 42. However, reliance upon a single clinical correlate 46 may not provide effective selection, since a given single clinical correlate may provide limited information on only a (possibly small) sub-set of the possible transmission pathways. Improved selection may be obtained by repeating the process for more clinical correlates, assuming such are available. The procedure just described can be repeated for additional correlates, such as location, equipment, and procedure, and a p-value can be computed for each of them to indicate the statistical likelihood of each transmission tree given the clinical correlate. Computational efficiency may optionally be improved by re-using the plurality of random trials 52 for computing the p-values for each clinical correlate. The p-values for the different clinical correlates can be combined into one p-value score by multiplying them together. This approach for combining the p-values assumes that the clinical correlates are statistically independent. If it is believed that the clinical correlates are not independent (e.g. location and caretaker are correlated), an alternative approach is to display all the p-value scores separately, instead of combining p-values by multiplication which assumes independence of random variables.

All clinical correlates that are available to the clinician may advantageously be thusly utilized in selecting the optimum transmission tree from amongst the plurality of transmission trees 42. The clinical correlates can include (but are not limited to) one or more of: location history, caretaker/healthcare provider history, equipment usage history, procedure history, patient symptoms, pathogen characteristics, and any other data that can be obtained that may be indicative of transmissions. Pathogen characteristics in this context may, by way of non-limiting example, include one or more of: multilocus sequence typing (MLST) type, antibiotic resistance profile, or so forth. The number of trials (N in the notation used above) can be set based on the desired level of accuracy needed to compute a p-value, while considering the running time needed to compute the p-value. N=1000 trials may be a good default value for number of random trials in order to obtain accurate estimates of the p-value, but this is merely a non-limiting illustrative example.

While the p-value is employed in the illustrative example as a metric for the statistical likelihood of significance of a transmission tree, other metrics of statistical likelihood may be employed, such as other null hypothesis metrics (Pearson's chi-squared test, et cetera).

In the foregoing approach, the goal is to select the optimal transmission tree from amongst the plurality of transmission trees 42 based on the estimated statistical likelihoods (illustrative p-values) of the trees given the clinical correlate. The selected optimal transmission tree is suitably displayed on the display 32 of the computer 30 (see FIG. 1).

The foregoing approach performs comparison of the transmission trees however, it is similarly contemplated to assess statistical likelihoods of individual parent-child infectious transmission links between pairs of HAI infected persons that occur in the transmission trees, in order to identify low confidence links. In this task, statistical likelihoods of parent-child infectious transmission links in the transmission trees may be computed based on correlation with one or more clinical correlates, or based on frequency of occurrence in the plurality of transmission trees (that is, a link that is inferred in a large fraction of the plurality of transmission trees 42 is statistically more likely to be an actual transmission pathway versus an outlier link that occurs in only one transmission tree), or based on a combination of correlation with one or more clinical correlates and frequency of occurrence in the plurality of transmission trees. (Where correlation with clinical correlates is employed in assessing statistical likelihood of individual links, the statistical likelihood computation may be repeated for a plurality of different clinical correlates, and the one or more low confidence links are identified based on the computed statistical likelihoods for the plurality of different clinical correlates.) One or more low confidence parent child infectious transmission links are identified based on the computed statistical likelihoods of the links. In this case, the transmission tree is displayed (e.g. the optimal transmission tree selected based on estimated p-values as previously described), with graphical indication of the one or more low confidence parent-child infectious transmission links in the display of the optimal transmission tree.

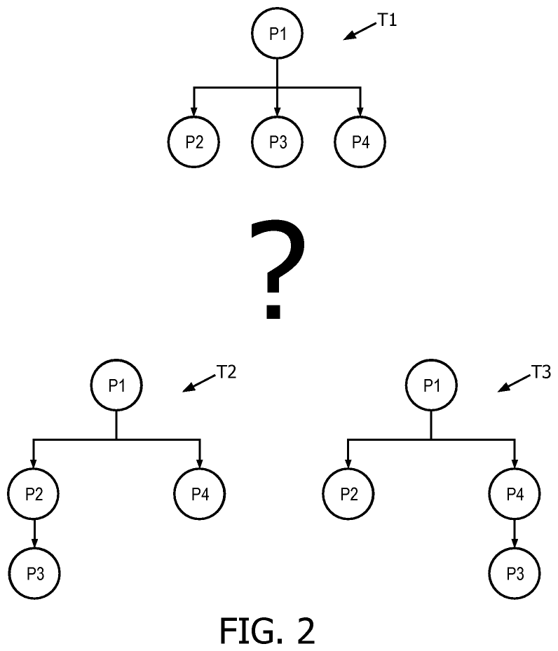

With reference to FIG. 2, in a common situation, low confidence links occur in groups. For example, FIG. 2 illustrates a situation in which the node P3 (corresponding to a particular HAI infected person) is not strongly linked to any other node. Thus, it may be likely that in one transmission tree T1 inferred by one transmission tree inference algorithm process, the node P3 is inferred to be a child from node P1 (that is, the person corresponding to node P1 is inferred to have transmitted the HAI to the person corresponding to node P3). In another transmission tree T2 inferred by another transmission tree inference algorithm process, the node P3 is inferred to be a child from node P2 (that is, the person corresponding to node P2 is inferred to have transmitted the HAI to the person corresponding to node P3). In yet another transmission tree T3 inferred by yet another transmission tree inference algorithm process, the node P3 is inferred to be a child from node P4 (that is, the person corresponding to node P4 is inferred to have transmitted the HAI to the person corresponding to node P3).

If the statistical link confidence is computed solely based on frequency of occurrence of each link in the plurality of transmission trees, then all three of the links P1→P3 in transmission tree T2, and the link P2→P3 in transmission tree T2, and the link P4→P3 in transmission tree T3, will be identified as low confidence links based on their respective computed statistical likelihoods. This is the case since each of the links P1→P3 and P2→P3 and P4→P3 occurs in only one transmission tree. (By contrast, the link P1→P2 occurs in all three transmission trees T1, T2, T3; and similarly the link P1→P4 occurs in all three transmission trees T1, T2, T3; hence, these links would have higher confidence).

In the case where the statistical likelihoods of the links are computed solely based on correlation with one or more clinical correlates, it may be that one of the three “candidate” links for node P3 has stronger correlation with the clinical correlate(s) than the other two “candidate” links. For example, the link P2→P3 in tree T2 may have stronger correlation with the clinical correlates than the lines P1→P3 and P4→P3 in trees T1, T3 respectively. In this case, a merger of the portion of the trees T1, T2, T3 involving node P3 may be performed which selects link P2→P3 over the other two, lower confidence links. On the other hand, if all three links involving node P3 have low statistical correlation with the statistical correlate(s), then the situation is again that all three of the links P1→P3 in transmission tree T2, and the link P2→P3 in transmission tree T2, and the link P4→P3 in transmission tree T3, will be identified as low confidence links.

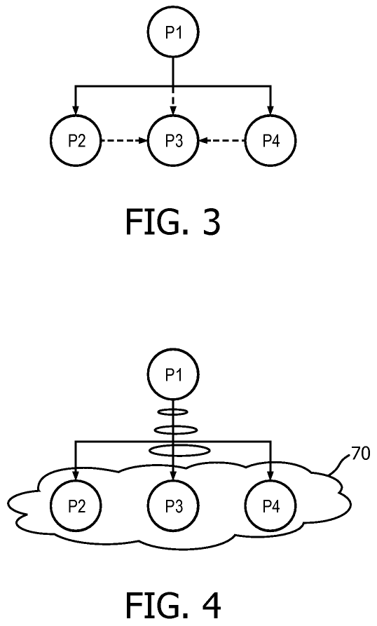

With reference now to FIGS. 3 and 4, in the case where all three of the links P1→P3 and P2→P3 and P4→P3 are found to be low confidence links, then the optimal transmission tree is preferably displayed with graphical indication of the low confidence parent-child infectious transmission links in the display of the optimal transmission tree. FIGS. 3 and 4 illustrate two contemplated approaches. In the example of FIG. 3, all three of the low confidence links P1→P3 and P2→P3 and P4→P3 are shown in the display of the transmission tree, but using dotted or dashed lines. The user can then readily identify that these links are of low confidence, and moreover since the node P3 has three such low confidence links connected with it, the user recognizes that the node P3 corresponds to the HAI infected person whose infectious pathway is uncertain. FIG. 4 illustrates another approach, in which the transmission tree T1 is chosen as the optimal tree and its link P1→P3 is included; however, the alternative low confidence links P2→P3 and P4→P3 are graphically indicated by grouping together the two or more low-confidence parent-child infectious transmission links using a graphical grouping annotation 70.

As an additional or alternative approach, the low confidence parent-child infectious transmission link(s) may be graphically indicated in the display of the transmission tree by labeling each low confidence link with a value or annotation indicative of its computed statistical likelihood, e.g. labeled with the count of the number of transmission trees of the plurality of transmission trees 42 that include the link, or labeled by that value normalized by the number of transmission trees (denoted as K in FIG. 1).

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory storage medium storing instructions readable and executable by an electronic processor to perform a healthcare associated infection (HAI) outbreak tracking method comprising:
performing a plurality of transmission tree inference algorithm processes operating on genetic variants data for a set of HAI infected persons to generate a plurality of transmission trees representing parent-child infectious transmission links between pairs of HAI infected persons;
for each transmission tree, computing the value of a correlation metric measuring correlation of the transmission tree with a clinical correlate; wherein the correlation metric comprises a count of parent-child infectious transmission links between pairs of HAI infected persons that match with the clinical correlate; and wherein the clinical correlate comprises clinical data that can be correlate with HAI transmission;
for each random trial of a plurality of random trials each comprising parent-child links randomly generated between pairs of HAI infected persons of the set of HAI infected persons, computing the value of the correlation metric;
estimating a statistical likelihood of each transmission tree given the clinical correlate from the computed values of the correlation metric for the random trials and for the transmission tree; wherein the estimated statistical likelihood for each transmission tree comprises a p-value for the transmission tree estimated as a fraction of the random trials whose correlation with the clinical correlate as measured by the correlation metric is higher than the correlation of the transmission tree with the clinical correlate as measured by the correlation metric; and
displaying at least one transmission tree of the plurality of transmission trees wherein the displayed at least one transmission tree is at least one of (i) selected for display based on the estimated statistical likelihoods or (ii) labeled with the estimated statistical likelihoods.

2. The non-transitory storage medium of claim 1 further comprising:
selecting an optimal transmission tree from amongst the plurality of transmission trees based on the estimated statistical likelihoods of the trees given the clinical correlate;
wherein the displaying includes displaying the optimal transmission tree.

3. The non-transitory storage medium of claim 2 wherein:
the estimated statistical likelihood for each transmission tree comprises a p-value for the transmission tree estimated as a fraction of the random trials whose correlation with the clinical correlate as measured by the correlation metric is higher than the correlation of the transmission tree with the clinical correlate as measured by the correlation metric; and
the optimal transmission tree is selected as the transmission tree having lowest p-value.

4. The non-transitory storage medium of claim 2 wherein:
the computing of the value of the correlation metric for each transmission tree, the computing of the value of the correlation metric for each random trial, and the estimating of the statistical likelihood of each transmission tree given the clinical correlate are repeated for a plurality of different clinical correlates; and
the optimal transmission tree is selected based on the estimated statistical likelihoods of the trees given the clinical correlates of the plurality of different clinical correlates.

5. The non-transitory storage medium of claim 4 wherein:
the estimated statistical likelihoods comprise p-values each estimated as a fraction of the random trials whose correlation with the clinical correlate as measured by the correlation metric is higher than the correlation of the transmission tree with the clinical correlate as measured by the correlation metric;
a composite p-value is computed for each transmission tree as a product of the p-values estimated for the transmission tree for the plurality of clinical correlates; and
the optimal transmission tree is selected as the transmission tree having lowest composite p-value.

6. The non-transitory storage medium of claim 1 wherein:
the estimated statistical likelihood for each transmission tree comprises a p-value for the transmission tree estimated as a fraction of the random trials whose correlation with the clinical correlate as measured by the correlation metric is higher than the correlation of the transmission tree with the clinical correlate as measured by the correlation metric;
the computing of the value of the correlation metric for each transmission tree, the computing of the value of the correlation metric for each random trial, and the estimating of the p-value of each transmission tree given the clinical correlate are repeated for a plurality of different clinical correlates; and
the displaying comprises displaying one or more transmission trees each labeled with the p-values of the transmission tree for the clinical correlates of the plurality of different clinical correlates.

7. The non-transitory storage medium of claim 1 wherein the clinical correlate comprises location history, caretaker/healthcare provider history, equipment usage history, procedure history, patient symptoms, or pathogen characteristics.

8. The non-transitory storage medium of claim 1 wherein the plurality of transmission tree inference algorithm processes include at least one inference algorithm process employing infection dates for the HAI infected persons as constraints on the transmission tree inference algorithm.

9. The non-transitory storage medium of claim 1 wherein the plurality of transmission tree inference algorithm processes include at least two transmission tree inference algorithm processes employing the same transmission tree inference algorithm but different tuning values for the transmission tree inference algorithm.

10. The non-transitory storage medium of claim 1 further comprising selecting the number of random trials based on a known or suspected pathogen causing the HAI.

11. The non-transitory storage medium of claim 1 further comprising:
estimating statistical likelihoods of parent-child infectious transmission links between pairs of HAI infected persons based on frequency of occurrences of the links in the plurality of transmission trees;
wherein the displaying includes displaying the at least one transmission tree with links of low estimated statistical likelihood graphically indicated in the display.

12. A device for performing healthcare associated infection (HAI) outbreak tracking, the device comprising:
a computer;
a display operatively connected with the computer; and
the non-transitory storage medium of claim 1, wherein the computer is operatively connected to read and execute the instructions stored on the non-transitory storage medium to perform the HAI outbreak tracking method.

13. A device for performing healthcare associated infection (HAI) outbreak tracking, the device comprising:
a computer;
a display operatively connected with the computer; and
a non-transitory storage medium storing instructions readable and executable by the computer to perform an HAI outbreak tracking method including:
performing a plurality of transmission tree inference algorithm processes operating on genetic variants data for a set of HAI infected persons to generate a plurality of transmission trees representing parent-child infectious transmission links between pairs of HAI infected persons;
computing statistical likelihoods of parent-child infectious transmission links in the transmission trees based on at least one of correlation with one or more clinical correlates and frequency of occurrence of the links in the plurality of transmission trees;
identifying one or more low confidence parent-child infectious transmission links based on the computed statistical likelihoods; and
displaying, on the display, at least one transmission tree selected from or derived from the plurality of transmission trees wherein the displaying includes graphically indicating the one or more low confidence parent-child infectious transmission links in the display of the at least one transmission tree.

14. The device of claim 13 wherein:
the computing of statistical likelihoods is repeated for a plurality of different clinical correlates; and
the one or more low confidence parent-child infectious transmission links are identified based on the computed statistical likelihoods for the plurality of different clinical correlates.

15. The device of claim 13 wherein the display of the at least one transmission tree uses solid lines to connect nodes representing pairs of HAI infected persons except that the one or more low confidence parent-child infectious transmission links are indicated at least by using dotted or dashed lines to connect the nodes representing the pairs of HAI infected persons of the low-confidence parent-child infectious transmission links.

16. The device of claim 13 wherein two or more low confidence parent-child infectious transmission links that form alternative possible links are indicated at least by grouping together the two or more low-confidence parent-child infectious transmission links using a graphical grouping annotation.

17. The device of claim 13 wherein the one or more low confidence parent-child infectious transmission links are indicated in the display of the at least one transmission tree by labeling each low confidence link with a value or annotation indicative of its computed statistical likelihood.

18. A method of healthcare associated infection (HAI) outbreak tracking comprising the operations:
(i) performing a plurality of transmission tree inference algorithm processes operating on genetic variants data for a set of HAI infected persons to generate a plurality of transmission trees representing parent-child infectious transmission links between pairs of HAI infected persons;
(ii) for each transmission tree, computing the value of a correlation metric measuring correlation of the transmission tree with a clinical correlate; wherein the correlation metric comprises a count of parent-child infectious transmission links between pairs of HAI infected persons that match with the clinical correlate; and wherein the clinical correlate comprises clinical data that can be correlated with HAI transmission;
(iii) for each random trial of a plurality of random trials each comprising parent-child links randomly generated between pairs of HAI infected persons of the set of HAI infected persons, computing the value of the correlation metric;
(iv) estimating a statistical likelihood of each transmission tree given the clinical correlate from the computed values of the correlation metric for the random trials and for the transmission tree; wherein the estimated statistical likelihood for each transmission tree comprises a p-value for the transmission tree estimated as a fraction of the random trials whose correlation with the clinical correlate as measured by the correlation metric is higher than the correlation of the transmission tree with the clinical correlate as measured by the correlation metric;
(v) selecting an optimal transmission tree from amongst the plurality of transmission trees based on the estimated statistical likelihoods of the trees given the clinical correlate; and
(vi) displaying the optimal transmission tree on a display;
wherein the operations (i), (ii), (iii), (iv), and (v) are performed by a computer executing instructions stored on a non-transitory storage medium.

19. The method of claim 18 wherein:
the operation (iv) comprises a p-value for the transmission tree estimated as a fraction of the random trials whose correlation with the clinical correlate as measured by the correlation metric is higher than the correlation of the transmission tree with the clinical correlate as measured by the correlation metric; and
the operation (v) comprises selecting the optimal transmission tree as the transmission tree having lowest estimated p-value.

20. The method of claim 19 wherein:
the operations (i), (ii), and (iv) are repeated for a plurality of different clinical correlates and a composite p-value is computed for each transmission tree as the product of the p-values estimated for the transmission tree for the different clinical correlates; and
the operation (v) comprises selecting the optimal transmission tree as the transmission tree having the lowest composite p-value.
